SEQUENCE LISTING (4 sequences)

SEQ ID NO: 1 - DNA, 62 bp, Artificial sequence
  Description of the artificial sequence Primer N in
gttataaatttggagtgtgaaggttattgcgtgcatattaccccagacaccccatcttatcg

SEQ ID NO: 2 - DNA, 31 bp, Artificial sequence
  Description of the artificial sequence Primer N out
ttgggcccgggctcaattaatcaggctcatc

SEQ ID NO: 3 - DNA, 31 bp, Artificial sequence
  Description of the artificial sequence Primer C out
gaggcccgggtatccaacgttttctcaaacg

SEQ ID NO: 4 - DNA, 62 bp, Artificial sequence
  Desciption of the artificial sequence Primer C in
cacgcaataaccttcacactccaaatttataactaatcttcctcttctgcaaaccctcgtgc

(19) United States
(12) Patent Application Publication
(10) Pub. No.: US 2006/0234358 A1
(43) Pub. Date: Oct. 19, 2006

(54) Method for the microbial production of aromatic amino acids and other metabolites of the aromatic amino acid biosynthetic pathway

(76) Inventors: Britta Anderlei, Aachen (DE); Georg Sprenger, Backnang-Maubach (DE); Hermann Sahm, Julich (DE); Johannes Josef Bongaerts, Pulheim (DE)

Correspondence Address: MORRISON & FOERSTER LLP, 12531 HIGH BLUFF DRIVE, SUITE 100, SAN DIEGO, CA, 92130-2040, US

(21) Appl. No.: 10/513,424

(22) PCT Filed: Apr. 29, 2003
(86) PCT No.: PCT/DE03/01380

§ 371 Date: Apr. 18, 2006

(30) Foreign Application Priority Data
May 2, 2002 (DE) 102 19 714.8

(51) Int. Cl. C12P 13/22; C12N 15/74; C12N 1/21

(52) U.S. Cl. 435/108; 435/252.33; 435/488

(57) ABSTRACT

A process for the microbial production of aromatic amino acids and other metabolites of the aromatic amino acid biosynthetic pathway The invention relates to a process for the microbial production of aromatic amino acids and other metabolites of the aromatic amino acid biosynthetic pathway. Microbially produced substances such as fine chemicals, in particular aromatic amino acids or metabolites of the aromatics biosynthetic pathway, are of great economic interest, and the need for amino acids, for example, continues to increase. The inventors found that, after introducing a pyc gene sequence into microorganisms, it was possible to produce aromatic amino acids and also metabolites of the aromatic biosynthetic pathway in an improved manner. The process of the invention is particularly suitable for producing L-phenylalanine.

The invention relates to a process for the microbial production of aromatic amino acids and other metabolites of the aromatic amino acid biosynthetic pathway.

Microbially produced substances such as fine chemicals, in particular aromatic amino acids or metabolites of the aromatics biosynthetic pathway, are of great economic interest, and the need for amino acids, for example, continues to increase. Thus, for example, L-phenylalanine is used for the preparation of medicaments and, in particular, also in the preparation of the sweetener aspartame (α-L-aspartyl-L-phenylalanine methyl ester). L-Tryptophan is needed as a medicament and a feedstuff supplement; L-tyrosine is likewise needed as a medicament and also as raw material in the pharmaceutical industry. Apart from isolation from natural materials, biotechnological production is a very important method in order to obtain amino acids in the desired optically active form under economically justifiable conditions. Biotechnological production is carried out either enzymatically or with the aid of microorganisms.

The latter, microbial production has the advantage of it being possible to use simple and inexpensive raw materials. However, since amino acid biosynthesis in the cell is controlled in multiple ways, a large variety of experiments to increase product formation have been undertaken previously. Thus, for example, amino acid analogs have been used in order to switch off biosynthetic regulation. For example, selection for resistance to phenylalanine analogs produced Escherichia coli mutants which made increased L-phenylalanine production possible (GB-2,053,906). A similar strategy also resulted in overproducing strains of Corynebacterium (JP-1903711976 and JP-39517/1978) and Bacillus (EP 0,138,526).

Furthermore, microorganisms constructed by means of recombinant DNA techniques are known in which biosynthetic regulation has likewise been eliminated by cloning and expressing the genes which code for key enzymes which are no longer feedback inhibited. EP 0,077,196 describes, as an example, a process for producing aromatic amino acids, which comprises overexpressing a no longer feedback-inhibited 3-deoxy-D-arabinoheptulosonate 7-phosphate synthase (DAHP synthase) in E. coli. EP 0,145,156 describes an E. coli strain which additionally overexpresses chorismate mutase/prephenate dehydratase to produce L-phenylalanine.

Said strategies share the fact that the intervention for improving production is limited to the biosynthetic pathway specific for the aromatic amino acids.

Production may be further increased, however, also by improved provision of the primary metabolites phosphoenolpyruvate (PEP) and erythrose 4-phosphate (Ery4P) required for producing aromatic amino acids. PEP is an activated precursor of the glycolytic product pyruvate (pyruvic acid); Ery4P is an intermediate of the pentose phosphate pathway.

The production of aromatic amino acids or of other metabolites of the aromatics biosynthetic pathway requires the primary metabolites phosphoenolpyruvate (PEP) and erythrose 4-phosphate (Ery4P) for condensation to give 3-deoxy-D-arabinoheptulosonate 7-phosphate (DAHP).

The effect of improved provision of the cellular primary metabolite phosphoenolpyruvate from glycolysis has already been investigated previously. Thus, transketolase overexpression, achieved by recombinant techniques, is known to be able to increase the amount of erythrose-4-P provided and, subsequently, to improve product formation of L-tryptophan, L-tyrosine or L-phenylalanine (EP 0,600,463).

Flores et al. (Flores et al. 1996. Nature Biotechnology 14:620-623) demonstrated that a spontaneous glucose-positive revertant of a sugar phosphotransferase system (PTS)-negative Escherichia coli mutant transported glucose via the galactose permease (GaIP) system into the cells and was capable of growing on glucose. Additional expression of the transketolase gene (tktA) leads to the observation of increased formation of the intermediate DAHP (Flores et al. Nature Biotechnology 14 (1996) 620-623). Further improvements in providing precursor metabolites for the aromatic amino acid biosynthesis pathway and improvements in the flux in the aromatic amino acid biosynthetic pathway are known to the skilled worker, for example from Bongaerts et al. (Bongaerts et al. Metabolic Engineering 3 (2001) 289-300).

The literature furthermore describes several strategies for increasing PEP availability, for example by means of a PEP-independent sugar uptake system in which, for example, the sugar phosphotransferase system (PTS) is completely inactivated and then replaced with a galactose permease or the genes glf (glucose-facilitator protein) and glk (glucokinase) from Zymomonas mobilis (Frost and Draths Annual Rev. Microbiol. 49 (1995), 557-579; Flores et al. Nature Biotechnology 14 (1996) 620-623; Bongaerts et al. Metabolic Engineering 3 (2001) 289-300).

Earlier patent applications (DE 19644566.3; DE 19644567.1; DE 19818541 A1; U.S. Pat. No. 6,316,232) also demonstrated that it was possible for substances of the aromatic biosynthetic pathway to be provided to an increased extent by increasing the enzyme activities of, for example, transketolase, transaldolase, glucose dehydrogenase or glucokinase in Escherichia coli or by combining the enzymes mentioned and a PEP-independent transport system.

In a number of microorganisms, pyruvate carboxylase plays an important part in the synthesis of those amino acids derived from the tricarboxylic acid cycle (TCA cycle).

The physiological role of pyruvate carboxylase is the anaplerotic reaction which, starting from pyruvate and CO₂ (or hydrogen carbonate), provides C4 bodies (oxaloacetate) (Jitrapakdee and Wallace, Biochemical Journal 340 (1999) 1-16). Oxaloacetate may be further metabolized by reacting with acetyl-CoA in the tricarboxylic acid cycle (e.g. also to give the amino acids glutamate and glutamine), or may provide, by way of transamination to give aspartic acid, precursors of the aspartate amino acid family (aspartate, asparagine, homoserine, threonine, methionine, isoleucine and lysine) (Peters-Wendisch et al. J. Mol. Microbiol. Biotechnol. 3 (2001) 295-300). Thus various groups were able to show that the activity of a pyruvate carboxylase plays a part in producing amino acids of the aspartate family in corynebacteria (DE 19831609; EP 1,067,192; Peters-Wendisch et al. Journal of Molecular Microbiology and Biotechnology 3 (2001) 295-300; Sinskey et al. U.S. Pat. No. 6,171,833 or WO 00/39305). WO 01/04325, for example, describes a fermentative process for producing L-amino acids of the aspartate amino acid family, using coryneform microorganisms containing a gene from the group consisting of dapA (dihydrodipicolinate synthase), lysC (aspartate kinase), gap (glycerolaldehyde 3-phosphate dehydrogenase), mqo (malate quinone oxidoreductase), tkt (transketolase), gnd (6-phosphogluconate dehydro-genase), zwf (glucose 6-phosphate dehydrogenase), lysE (lysine export), zwa1 (unnamed protein product), eno (enolase), opcA (putative oxidative pentose phosphate cycle protein) and also a pyc gene sequence (pyruvate carboxylase). In this connection, the aromatic amino acid L-tryptophan is likewise mentioned as a product of the process described in WO 01/04325, in addition to the amino acids of the aspartate amino acid family. It is, however, not indicated which special gene sequence or which special enzyme is suitable for specific production of aromatic amino acids and metabolites of the aromatic amino acid biosynthetic pathway.

Pyruvate carboxylase genes (pyc genes) have been isolated from a number of microorganisms, characterized and expressed in recombinant form. Thus, pyruvate carboxylase genes have been detected previously in bacteria such as corynebacteria, rhizobia, brevibacteria, Bacillus subtilis, mycobacteria, Pseudomonas, Rhodopseudomonas spheroides, Camphylobacter jejuni, Methanococcus jannaschii, in the yeast Saccharomyces cerevisiae and in mammals such as humans (Payne & Morris J Gen. Microbiol. 59 (1969) 97-101; Peters-Wendisch et al. Microbiology 144 (1998) 915-927; Gokarn et al. Appl. Microbiol. Biotechnol. 56 (2001) 188-195; Mukhopadhyay et al. Arch. Microbiol. 174 (2000) 406414; Mukhopadhyay & Purwantini, Biochim. Biophys. Acta 1475 (2000) 191-206; Irani et al. Biotechnol. Bioengin. 66 (1999) 238-246; U.S. Pat. No. 6,171,833; Dunn et al. Arch. Microbiol 176 (2001) 355-363; Dunn et al. J. Bacteriol. 178 (1996) 5960-5970; Jitrapakdee et al. Biochem. Biophys. Res. Comm. 266 (1999) 512-517; Velayudhan & Kelly Microbiology 148 (2002) 685-694; Mukhopadhyay et al. Arch. Microbiol. 174 (2000) 406414; EP 1,092,776).

No pyruvate carboxylases have been described previously from Escherichia coli and other enterobacteria.

Recently, it was demonstrated in recombinant Escherichia coli or Salmonella typhimurium cells carrying the Rhizobium etli pyc gene that pyruvate carboxylase expression distinctly altered the product spectrum of said cells, to be precise toward the C4 bodies (e.g. succinate), with pyruvate-derived substances such as lactate or acetate being reduced (Gokarn et al. Biotechnol. Letters 20 (1998) 795-798; Gokarn et al. Applied Environm. Microbiol. 66 (2000) 1844-1850; Gokarn et al. Appl. Microbiol. Biotechnol. 56 (2001) 188-195; Xie et al. Biotechnol. Letters 23 (2001) 111-117). Expression of a Bacillus subtilis pyc gene in E. coli achieved formation of the L-amino acids threonine, glutamic acid, homoserine, methionine, arginine, proline and isoleucin (EP 1,092,776). An increased formation of aromatic amino acids or of metabolites of the aromatic biosynthetic pathway has not been described in the literature (Xie et al. Biotechnol. Letters 23 (2001) 111-117; Gokarn et al. Biotechnol. Letters 20 (1998) 795-798; Gokarn et al. Applied Environm. Microbiol. 66 (2000) 1844-1850; Gokarn et al. Appl. Microbiol. Biotechnol. 56 (2001) 188-195; EP 1,092,776).

It is therefore the object of the invention to provide a process which can be used to produce aromatic amino acids and other metabolites of the aromatic amino acid biosynthetic pathway.

Starting from the preamble of claim 1, the object is achieved according to the invention by the features indicated in the characterizing part of claim 1.

Advantageous further embodiments of the invention are indicated in the dependent claims.

It is now possible, using the process of the invention, to produce microbially aromatic amino acids and also metabolites of the aromatic amino acid biosynthetic pathway.

The process of the invention is particularly suitable for producing L-phenylalanine.

Aromatic amino acids and other metabolites of the aromatic amino acid biosynthetic pathway, also referred to as “substances” hereinbelow, mean for the purpose of the invention in particular the aromatic amino acids L-phenylalanine, L-tryptophan and L-tyrosine. The term metabolites from the aromatic amino acid biosynthetic pathway may also mean compounds derived from 3-deoxy-D-arabinoheptulosonate 7-phosphate (DAHP), such as, for example, D-arabinoheptulosonate (DAH), shikimic acid, chorismic acid and all of their derivatives, cyclohexadiene-trans-diols, indigo, indoleacetic acid, adipic acid, melanine, quinones, benzoic acid and also potential derivatives and secondary products thereof. It should be noted here that production of indigo, adipic acid and other unnatural secondary products requires, in addition to the interventions of the invention, further genetic modifications on the microorganisms producing said substances. However, this should include all compounds whose biochemical synthesis is promoted by providing increased amounts of PEP.

Surprisingly, the inventors found that, after introducing a pyc gene sequence into microorganisms which naturally have no pyruvate carboxylase, or after amplifying a pyc gene sequence present, it was possible to produce aromatic amino acids and also metabolites of the aromatic biosynthetic pathway in an improved manner.

Within the scope of the present invention, all gene sequences coding for a pyruvate carboxylase are referred to by the generic term “pyc gene sequence” hereinbelow.

The term “introducing” thus means, within the scope of the present invention, any process steps which result in inserting a pyc gene sequence in microorganisms having no pyc gene sequence. Furthermore, however, the term “introducing” may also mean amplification of a pyc gene sequence already present.

A number of different detection methods have been described for the enzymatic activity of pyruvate carboxylases. The test principle is detection of the oxaloacetate produced from pyruvate. The enzyme pyruvate carboxylase catalyzes the carboxylation of pyruvate, forming oxaloacetate in the process. The activity of a pyruvate carboxylase depends on biotin as a prosthetic group on the enzyme and also depends on ATP and magnesium ions. In the first reaction step, ATP is cleaved to give ADP and inorganic phosphate and the enzyme-biotin complex is carboxylated by hydrogen carbonate. In the second step, the carboxyl group is transferred from the enzyme-biotin complex to pyruvate, forming oxaloacetate as a result.

Brevibacterium lactofermentum pyruvate carboxylase can be detected, for example, in crude extracts obtained by ultrasound treatment by carrying out coupled enzyme assays with malate dehydrogenase or citrate synthase which in each case serve to detect the oxaloacetate formed (Tosaka et al. Agric. Biol. Chem. 43 (1979) 1513-1519).

Methanococcus janaschii pyruvate carboxylase was detected by means of coupling with malate dehydrogenase (Mukhopadhyay et al. Arch. Microbiol. 174 (2000) 406-414).

In Corynebacterium glutamicum cells which had been permeabilized by means of hexadecyltrimethylammonium bromide (CTAB), pyruvate carboxylase activity was detected in a discontinuous process by coupling to a glutamate-oxaloacetate transaminase (Peters-Wendisch et al. Microbiology 143 (1997) 1095-1103).

Uy et al. (Journal of Microbiological Methods 39 (1999) 91-96) used, likewise in CTAB-permeabilized C. glutamicum cells, a discontinuous process in which the remaining pyruvate concentration was determined by means of lactate dehydrogenase and conversion of pyruvate and NADH to give lactate and NAD was determined fluorimetrically. In recombinant E. coli cells expressing the Rhizobium etli pyc gene sequence, the pyruvate carboxylase activity in crude extracts was determined by coupling with the enzyme citrate synthase and spectrophotometric coenzyme A detection at 412 nm via formation of thionitrobenzoate (Gokarn et al. Appl. Microbiol. Biotechnol. 56 (2001) 188-195; Payne & Morris J. Gen. Microbiol. 59 (1969) 97-101).

The activity of human pyruvate carboxylase and that of recombinant yeast pyruvate carboxylase were determined by fixation of radiolabeled ¹⁴C carbonate (Jitrapakdee et al. Biochem. Biophys. Res. Commun. 266 (1999) 512-517; Irani et al. Biotechnol. Bioengin. 66 (1999) 238-246).

Amplifying the pyruvate carboxylase activity or providing pyruvate carboxylase for the first time in microorganisms presumably causes increased intracellular availability of phosphoenolpyruvate (PEP) so that the latter is no longer consumed in anaplerotic reactions. This may then result in an improved microbial synthesis of substances derived from PEP, in particular aromatic amino acids and also other metabolites of the aromatic amino acid biosynthetic pathway. As the inventors demonstrated, introducing a pyc gene sequence into microorganisms resulted in an improved microbial synthesis of substances derived from PEP. In particular, DAHP or its degradation product, DAH, was increasingly found back in the culture supernatant if the second step of the aromatics biosynthetic pathway is blocked by a mutation of the aroB gene. DAHP which is synthesized by way of condensation of PEP and Ery4P forms the starting substance for aromatic amino acids and also other metabolites of the aromatic amino acid biosynthetic pathway. In the literature, DAHP and DAH are discussed as indicators for increased PEP availability (Frost and Draths Annual Rev. Microbiol. 49 (1995), 557-579; Flores et al. Nature Biotechnology 14 (1996) 620-623; Bongaerts et al. Metabolic Engineering 3 (2001) 289-300).

The term “amplification” of the pyc gene sequence describes, in the context of the present invention, the increase in pyruvate carboxylase activity. For this purpose, the following measures may be mentioned by way of example:
introducing the pyc gene sequence, for example by means of vectors or temperate phages;
increasing the number of gene copies coding for pyruvate carboxylase (pyc gene sequence), for example by means of plasmids, with the aim of introducing into the microorganism an increased number of copies of the pyc gene sequence, from a slightly increased (e.g. 2 to 5 times) to a greatly increased (e.g. 15 to 50 times) number of copies;
increasing gene expression of the pyc gene sequence, for example by increasing the rate of transcription, for example by using promoter elements such as, for example, Ptac, Ptet or other regulatory nucleotide sequences and/or by increasing the rate of translation, for example by using a consensus ribosome binding site;
adding biotin to the fermentation medium in order to better supply the cells with the prosthetic group biotin which is essential for pyruvate carboxylase or enhancing enzymes present which are capable of biotin biosynthesis, or introducing said enzymes into the microorganism.

Using inducible promoter elements, for example Iacl^{q}/Ptac, makes it possible to switch on new functions (induction of enzyme synthesis), for example by adding chemical inducers such as isopropylthiogalactoside (IPTG).

Alternatively, it is furthermore possible to overexpress the pyc gene sequence by altering the composition of the media and the course of the culturing. The addition of essential growth substances to the fermentation medium may also cause improved production of the substances for the purpose of the invention.

Expression is also improved by measures of extending the mRNA lifetime. Furthermore, preventing degradation of the enzyme protein also enhances enzyme activity. Increasing the endogenous activity of a pyruvate carboxylase present (e.g. in Bacillus subtilis or corynebacteria), for example by mutations which are generated in a nondirected manner according to classical methods, such as, for example, by UV radiation or mutation-causing chemicals, or by mutations which are generated specifically by means of genetic-engineering methods such as deletion(s), insertion(s) and/or nucleotide substitution(s). Combinations of said methods and of further, analogous methods may also be used for increasing pyruvate carboxylase activity.

The pyc gene sequence is preferably introduced by integrating the pyc gene sequence into a gene structure or into a plurality of gene structures, said pyc gene sequence being incorporated into the gene structure as a single copy or with an increased number of copies.

“Gene structure” means any gene or any nucleotide sequence carrying a pyc gene sequence. Appropriate nucleotide sequences may be, for example, plasmids, vectors, chromosomes, phages or other non-closed-circle nucleotide sequences. For example, the pyc gene sequence may be introduced into the cell on a vector or inserted into a chromosome or introduced into the cell via a phage. These examples are not intended to exclude other combinations of gene distributions from the invention. In the case that a pyc gene sequence is already present, the number of the pyc gene sequences contained in the gene structure should exceed the natural number.

The pyc gene sequence used for the process of the invention may be derived, for example, from Rhizobium (Gokarn et al. Appl. Microbiol. Biotechnol. 56 (2001) 188-195), Brevibacterium, Bacillus, Mycobacterium (Mukhopadhyay and Purwantini Biochimica et Biophysica Acta 1475 (2000) 191-206), Methanococcus (Mukhopadhyay et al. Arch. Microbiol. 174 (2000) 406414), Saccharomyces cerevisiae (Irani et al. Biotechnology and Bioengineering 66 (1999) 238-246) Pseudomonas, Rhodopseudomonas, Campylobacter or Methanococcus jannaschii (Mukhopadhyay et al. Arch. Microbiol. 174 (2000) 406-414). A pyc gene sequence from Corynebacterium strains, in particular from Corynebacterium glutamicum (Peters-Wendisch et al. Microbiology 144 (1998) 915-927; Peters-Wendisch et al. J. Mol. Microbiol. Biotechnol. 3 (2001) 295-300, has proved advantageous. Genes for pyruvate carboxylases from other organisms are also suitable. The skilled worker appreciates that further pyc gene sequences are identifiable from generally accessible databases (such as, for example, EMBL, NCBI, ERGO) and are clonable from such other organisms by means of gene cloning techniques, for example using the polymerase chain reaction PCR.

The process of the invention makes use of microorganisms into which a pyc gene sequence has been introduced in a replicable form. Suitable microorganisms for transformation with a pyc gene sequence are organisms of the family of Enterobacteriaceae such as, for example, Escherichia species, but also microorganisms of the genera Serratia, Bacillus, Corynebacterium or Brevibacterium and other strains known from classical amino acid processes. Escherichia coli is particularly suitable.

According to the requirements of the Budapest Treaty, the following strain was deposited with the DSMZ on March 22, 2002: Escherichia coli K-12 LJ110 aroB/pF36, under the number DSM 14881.

The microorganisms or host cells may be transformed by means of chemical methods (Hanahan D, J. Mol. Biol. 166 (1983) 557-580) and also by electroporation, conjugation, transduction or by subcloning from plasmid structures known in the literature. In the case of cloning pyruvate carboxylase from Corynebacterium glutamicum, for example, the polymerase chain reaction (PCR) method is suitable, for example, for directed amplification of the pyc gene sequence with chromosomal DNA from Corynebacterium glutamicum strains.

It is advantageous to use, for transformation, microorganisms in which one or more enzymes which additionally are involved in the synthesis of the aromatic amino acids and other metabolites of the aromatic amino acid biosynthesis pathway are deregulated and/or in which the activity of said enzymes is increased. Particular use is made of transformed cells capable of producing an aromatic amino acid which preferably is L-phenylalanine.

In a further advantageous embodiment of the process of the invention, it is possible, in microorganisms having a pyc gene sequence, to reduce or inactivate or completely switch off expression of the genes coding for enzymes which compete for PEP with pyruvate carboxylase, such as, for example, PEP carboxylase, the PEP-dependent sugar phosphotransferase system (PTS) or pyruvate kinases, individually or in combination, and to use said microorganisms. Thus it may be possible to improve further the provision of PEP for the synthesis of aromatic amino acids and other metabolites of the aromatic amino acid biosynthetic pathway and thereby improve production of said compounds.

This advantageous embodiment also comprises increasing the activity of a transport protein for PEP-independent uptake of glucose into microorganisms which have a PEP-dependent transport system for glucose and which are employed in the process of the invention. The additional integration of a PEP-independent transport system allows providing an increased amount of glucose in the microorganism producing the substances. PEP is not required as an energy donor for these reactions and is thus increasingly available, starting from a constant flux of substances in the glycolysis and the pentose phosphate pathway, for condensation with erythrose 4-phosphate (Ery4-P) to give the primary metabolite of the general biosynthetic pathway for aromatic compounds such as, for example, deoxy-D-arabinoheptulosonate 7-phosphate (DAHP) and, subsequently, for producing, for example, aromatic amino acids such as L-phenylalanine, tyrosine or tryptophan, for example.

The advantageous use of a PEP-independent sugar transport system, of a glucose-facilitator protein (Glf) and of the genes for transketolase, transaldolase and glucokinase has already been demonstrated in earlier patent applications (DE 19644566.3, DE 19644567.1, DE 19818541 A1; U.S. Pat. No. 6,316,232).

In a preferred embodiment, it is possible to use, in the process of the invention for producing substances, microorganisms in which one or more enzymes which are additionally involved in the synthesis of said substances are deregulated and/or in which the activity of said enzymes is increased. Said enzymes are particularly those of the aromatic amino acid metabolism and especially DAHP synthase (e.g. in E. coli AroF or AroH), shikimate kinase and chorismate mutase/prephenate dehydratase (PheA). Any other enzymes involved in the synthesis of aromatic amino acids or metabolites of the aromatic amino acid biosynthesis pathway and of secondary products thereof may also be used.

Apart from the pyc gene sequence, the deregulated and overexpressed DAHP synthase has proved to be particularly suitable for producing metabolites of the aromatic amino acid biosynthetic pathway and derivatives thereof, such as, for example, adipic acid, bile acid and quinone compounds, and also derivatives thereof. In order to increase synthesis of, for example, L-tryptophan, L-tyrosine, indigo, derivatives of hydroxy- and aminobenzoic acid and naphtho- and anthroquinones, and also secondary products thereof, shikimate kinase, in addition, should be deregulated and its activity be increased. In addition, a deregulated and overexpressed chorismate mutase/prephenate dehydratase is particularly important for efficient production of phenylalanine, phenylpyruvic acid and derivatives thereof. However, this should also include any other enzymes whose activities contribute to the microbial synthesis of metabolites other than those of the aromatic amino acid biosynthetic pathway, i.e. compounds whose production is promoted by providing PEP, for example CMP ketodeoxyoctulosonic acid, UDP N-acetylmuramic acid, or N-acetylneuraminic acid. Increasing the amount of PEP provided may, in this connection, not only have a beneficial effect on DAHP synthesis but also promote the introduction of a pyruvate group in the synthesis of 3-enolpyruvoylshikimate 5-phosphate as a precursor of chorismate. The production of indigo, adipic acid, cyclohexadiene-trans-diols and other unnatural secondary products requires, apart from the features of the process of the invention, further genetic modifications on the microorganisms producing said substances.

It is intended hereinbelow to indicate the materials and methods used and to illustrate the invention by way of experimental examples and comparative examples:

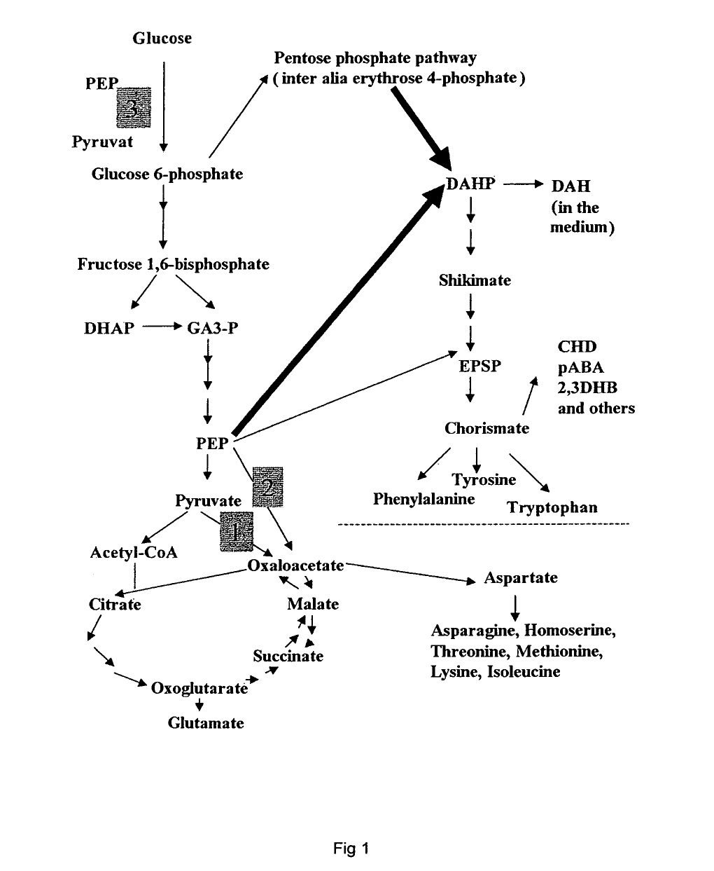

FIG. 1 depicts the linkages between the central metabolism and the aromatic amino acid biosynthetic pathway of bacteria, emphasizing the reactions of phosphoenolpyruvate and pyruvate. Reaction 1 indicates the pyruvate carboxylase reaction, reaction 2 the phosphoenolpyruvate carboxylase reaction and reaction 3 the PEP-dependent sugar phosphotransferase system (PTS).

| | |
|---|---|
| CHD = | cyclohexadiene-carboxylate-trans-diols |
| DAHP = | 3-deoxyarabinoheptulosonate 7-phosphate |
| DAH = | 3-deoxyarabinoheptulosonate |
| DHAP = | dihydroxyacetone phosphate |
| 2,3-DHB = | 2,3-dihydroxybenzoate |
| EPSP = | enolpyruvolylshikimate phosphate |
| GA3-P = | glyceraldehyde 3-phosphate |
| pABA = | para-aminobenzoate |
| PEP = | phosphoenolpyruvate |

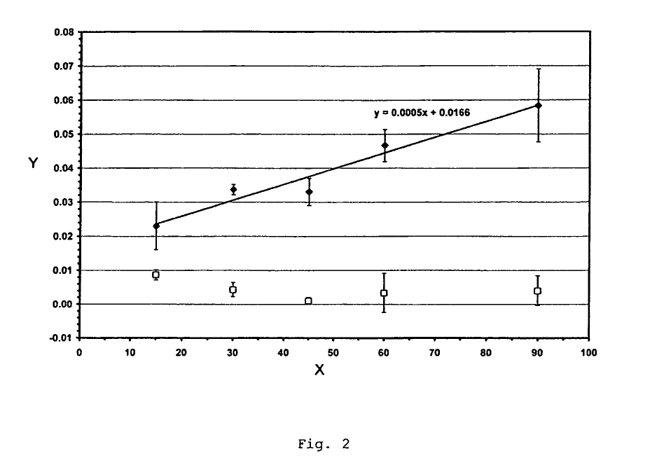

FIG. 2 depicts, by way of example, experimental data of the detection of pyruvate carboxylase activity. The abscissa X represents the time in seconds and the ordinate Y represents the extinction at a wavelength of 412 nm. The data points represented by diamonds filled with black are results obtained with E. coli cells transformed with a pyc vector. The data points represented by empty squares represent the results of the E. coli cells transformed with an empty vector without pyc gene sequence. The continuous grey line represents the regression line.

TABLE 1
Plasmids and bacteria strains used

| | | |
|---|---|---|
| Name | Relevant properties | Origin/reference |

Bacteria

| | | |
|---|---|---|
| Escherichia | Cloning strain | Hanahan D. J. Mol. Biol. |
| coli DH5α | | 166 (1983) 557-580 |
| Escherichia | | Escherichia coli K-12 |
| coli LJ110 | | wild-type |
| Escherichia | Defective for enzyme | Marco Krämer, PhD |
| coli | AroB | thesis, Univ. Düsseldorf, |
| LJ110aroB | | 1999; Jül-Bericht 3824 |
| Escherichia | Deletion of PEP- | Present study (see |
| coli | carboxylase gene | Example 1) |
| LJ110Δppc | | |

Plasmids

| | | |
|---|---|---|
| PVWEx1- | pyc gene sequence | Peters-Wendisch et al. |
| pyc | Kanamycin resistance | J. Mol. Microbiol. |
| | | Biotechnol. 3 (2001) 295-300 |
| PACYCPtac | laclq/Ptac Cm resistance | Siewe et al. 1996 |
| pF-36 | pACYCPtac Sphl + HindIII | Deposited with the DSMZ |
| | restricted plus 3.7 kb pyc | under reference number |
| | fragment from pVWEx1- | DSM 14881 |
| | pyc | |

### EXAMPLE 1

### Cloning of the pyc Gene Sequence, Expressing in Escherichia coli Strains

The first cloning of the pyc gene sequence from Corynebacterium glutamicum ATCC13032 has been described in Peters-Wendisch et al. Microbiology 144 (1998) 915-927. Subcloning of said pyc gene sequence into the expression vector pVWExl-pyc has been described in Peters-Wendisch et al. J. Mol. Microbiol. Biotechnol. 3 (2001) 295-300. A 3.7 kb DNA fragment containing the C. glutamicum pyc gene sequence was obtained from the pVWExl-pyc vector by means of restriction with the enzymes SphI and HindIII. This 3.7 kb fragment was ligated with the vector pACYCPtac (restricted with SphI plus HindIII). Transformation into the E. coli strain DH5α was carried out, followed by selection on LB plates containing chloramphenicol (25 mg/l). Plasmids containing the correct insert were referred to as pF36.

Mutations producing defects in the biosynthesis of aromatic amino acids were introduced by P1-mediated transduction. The defects of the two shikimate kinases (aroL and aroK) were generated by successive P1 transduction from the strain DV80 (aroK::kan, aroL::Tn10, Vinella et al. Journal of Bacteriology 178 (1996) 3818-3828). For this purpose, the E. coli K-12 LJ 10 wild-type strain was first selected for resistance to Kanamycin (retaining of the arok::Kan marker). A subsequent, second P1 transduction involved selection for retaining the tetracycline resistance marker (retaining of the aroL::Tn10 marker). Cells having both resistances were then checked for auxotrophy for the aromatic amino acids L-phenylalanine, L-tyrosine, L-tryptophan (in each case 40 mg/l) and for auxotrophy for p-aminobenzoic acid, p-hydroxybenzoic acid and 2,3-dihydroxybenzoic acid (in each case 20 mg/l). Mutants having a defect in aroB were obtained by carrying out a P1 transduction from the donor strain AB2847 rpe::Km aroB into the strain LJ110. The first step here involved selection for resistance to Kanamycin. Bacteria which were also auxotrophic for aromatic amino acids and for shikimate (aroB-negative) were used in the subsequent steps. A second P1 transduction (using a P1 lysate from the wild-type strain LJ110) involved selection for utilization of pentose sugars on minimal medium. The rpe::Km defect results in a pentose-negative phenotype, retaining rpe results in pentose utilization. Cells which became pentose-positive but remained auxotrophic for aromatics (aroB) continued to be used as LJ110 aroB (Marco Krämer, PhD thesis, Universität Düsseldorf, 1999, p. 34).

The strain LJ110 Appc was prepared by the crossover PCR method of Link et al. (Link et al. J. Bacteriol. 179 (1997) 6228-6237). The oligonucleotide primer pairs used for PCR amplification were: outer primer Nin 5′GTTATAAATTTGGAGTGTGAAGGTTATTGCGTGCATATTACCCCAGACACC CCATCTTATCG 3′ (Seq. ID. No.1) and inner primer Nout 5′TTGGGCCCGGGCTCMTTMTCAGGCTCATC 3′ (Seq. ID. No. 2) for the 5′ region upstream of the ppc gene. And for the 3′ region downstream of the ppc gene: outer primer Cout 5′GAGGCCCGGGTATCCMCGTTTTCTCAAACG 3′ (Seq. ID. No. 3) and inner primer Cin 5′CACGCMTMCCTTCACACTCCAAATTTATMCTMTCTTCCTCTTCTGCAAA CCCTCGTGC 3′ (Seq. ID. No.4). The DNA fragment generated by PCR contained special introduced cleavage sites for the restriction enzyme XmaCI. Cloning to the XmaCI site of the pKO3 vector generated an in-frame deletion of the ppc gene which was then introduced into the strain LJ110 by way of the method described (Link et al. Bacteriol. 179 (1997) 6228-6237). After selection for sucrose resistance, strains were obtained which are auxotrophic for addition of C4 substrates such as succinate or malate. The correct chromosomal deletion (Δppc) was confirmed by means of PCR with chromosomal DNA from said mutants. The correct mutants were referred to as LJ110Δppc.

### EXAMPLE 2

### Detection of Pyruvate Carboxylase Activity

Performing the enzymic pyruvate-carboxylase assay in recombinant Escherichia coli cells is described by way of example below.

Escherichia coli LJ110 Δppc cells which have been transformed either with the empty vector (control vector without pyc gene sequence) pACYCtac or with the pyc-containing vector pF36 were grown in a minimal medium (see preculture medium, Table 2) containing 0.5% glucose and chloramphenicol (25 mg/l). Biotin (200 μg/l) was added to the medium in order to meet the biotin requirement of pyruvate carboxylase. Since the cells have a defective PEP carboxylase, 0.5 g/l sodium succinate was added to the minimal medium. The cultures were incubated in shaker flasks (500 ml Erlenmeyer flasks with a volume of 100 ml) on a rotary shaker (200 revolutions per minute) at 37° C. for 6 hours, until they had reached an optical density at 600 nm (OD₆₀₀) of from 1 to 1.5 (late exponential growth phase). The pyruvate carboxylase was induced by adding IPTG to the culture media to give a final concentration of 100 μM. After reaching the optical density indicated, the cultures were harvested by centrifugation. The sediments thus obtained were washed twice with 100 mM TrisHCl buffer (pH 7.4). The cells were then resuspended in the same buffer and their concentration was adjusted so as to have an OD₆₀₀ of 5 in 1 ml of buffer. Such samples were admixed with 10 μl of toluene per ml and mixed on a Vortex instrument for 1 minute. This was followed by incubation at 4° C. (on ice) for 10 minutes. This resulted in the cells being permeabilized. 100 μl aliquots of said cells were then used for the subsequent pyruvate carboxylase assay.

The principle of the assay is detection of oxaloacetate (OAA), formed from pyruvate and hydrogen carbonate, via coupling with the auxiliary enzyme citrate synthase and acetyl-coenzyme A (Acetyl-CoA) according to the following reactions:
Pyruvate+HCO₃⁻+ATP→OAA+ADP+Pᵢ (1)
OAA+Acetyl-CoA→Citrate+HS-CoA⁻ (2)
HS-CoA+DTNB→CoA derivative+TNB²⁻ (3)
OAA=Oxaloacetate
DTNB=Dithionitrobenzoic acid
TNB²⁻=5-Thio-2-nitrobenzoate
CoA derivative=Mixed disulfide of CoA and thionitrobenzoic acid

Pyruvate carboxylase, Pyc, converts pyruvate with ATP hydrolysis to give oxaloacetate (OAA) (1). The OAA produced is reacted with acetyl-CoA via the citrate synthase reaction (2) to give citrate and coenzyme A (HS-CoA). Detection of Pyc activity is based on the reaction (3) of the coenzyme A (HS-CoA) being released with dithionitrobenzoic acid to give a mixed disulfide of CoA and thionitrobenzoic acid and a molar equivalent of yellow 5-thio-2-nitrobenzoate (TNB²⁻). The latter has a molar extinction coefficient of 13.6 mM⁻¹ cm⁻¹ and can be detected photometrically at a wavelength of 412 nm. The rate of TNB²⁻ formation correlates directly with OAA acetylation and thus with the conversion of pyruvate to OAA by pyruvate carboxylase.
1 ml of the assay mixture contained:
NaHCO₃ (25 mM), MgCl₂ (1 mM), Acetyl-CoA (0.2 mM), DTNB (0.2 mM), ATP (4 mM), citrate synthase (1 U=1 unit), cell suspension (0.5 OD₆₀₀), assay buffer (100 mM Tris-HCl pH 7.3). The mixtures were preheated to 25° C. in a 2 ml Eppendorf reaction vessel for 2 minutes. The reaction was started by adding pyruvate (5 mM).

The reaction was stopped at the appropriate points in time by transferring the reaction vessels to liquid nitrogen and, during the thawing process, the biomass was removed by centrifugation at 15,300 rpm at 4° C. Extinction at 412 nm was determined photometrically in the clear supematants. Mixtures without pyruvate were used as reference.

The data shown in FIG. 2 result in an increase in extinction [E₄₁₂] of 0.029 per minute and thus an absolute pyruvate carboxylase activity of 210 mU/ml. This results in a specific Pyc activity of 42 mU/OD₆₀₀, based on the number of cells used of OD₆₀₀=0.5. No Pyc activity was found in the controls.

### EXAMPLE 3

### Fermentation for Obtaining DAH, using Recombinant Pyruvate Carboxylase

The accumulation of DAH (degradation product of DAHP) as a first metabolite of the aromatics biosynthetic pathway may be detected by means of an aroB mutation. The strains E. coli K-12 LJ1 0 aroB/pF36 (=DSM 14881, “PYC”) and the control strain E. coli K-12 LJ 110 aroB/pACTCtac (empty vector “EV”) were used. The procedure was carried out in 6 Sixfors Vario laboratory fermenters (2 liters) connected in parallel and containing a volume of 1.5 l.

The studies were carried out using the following media compositions and under the following fermentation conditions:

TABLE 2
Preculture medium:

| | Concentration | |
|---|---|---|
| Substance | [g/l] | |
| KH₂PO₄ | 3 | |
| K₂HPO₄ | 12 | |
| (NH₄)₂SO₄ | 5 | |
| MgSO₄*7H₂O | 0.3 | |
| CaCl₂*2H₂O | 0.015 | |
| NaCl | 1 | |
| Glucose*1H₂O | 5 | |
| Citrate/FeSO₄*7H₂O | 0.1125 | |
| Thiamine | 0.0075 | |
| Tyrosine | 0.04 | |
| Trace elements | 1 | ml/l |
| Biotin | 0.0002 | |
| Chloramphenicol | 0.025 | |
| Tryptophan | 0.04 | |
| Phenylalanine | 0.04 | |
| Shikimate | 0.04 | |

TABLE 3
Fermentation medium:

| | Concentration | |
|---|---|---|
| Substance | [g/l] | |
| KH₂PO₄ | 3 | |
| (NH₄)₂SO₄ | 5 | |
| MgSO₄*7H₂O | 3 | |
| CaCl₂*2H₂O | 0.015 | |
| NaCl | 1 | |
| Glucose*1H₂O | 30 | |
| Citrate/FeSO₄*7H₂O | 0.1125 | |
| Thiamine | 0.0075 | |
| Tyrosine | 0.25 | |
| Trace elements | 1 | ml/l |
| Biotin | 0.0002 | |
| Chloramphenicol | 0.025 | |
| Tryptophan | 0.282 | |
| Phenylalanine | 0.228 | |
| Shikimate | 0.024 | |

Fermentation Conditions and Experimental Procedure
Fed batch (6 times parallel reaction mixture in a stirred and gassed “Sixfors-Vario” bioreactor from Infors, with off-gas analysis from Rosemount)
duration: 30 h
Temperature [° C.]: 37 (controlled)
pH: 6.5 (controlled)
pO₂: 30% (controlled)
titrant: 25% NH₃
inducer: IPTG (100 μmol/l), initially charged
starting volume: 1.5 l
starting conditions:
   number of stirrer revolutions: 500 rpm, flow rate 0.5 l/min
   in the growth phase, increase step by step the number of stirrer revolutions and the flow rate (max. 1.5 l/min), when reaching 900 to 1000 rpm, switch off pO₂ regulation via number of stirrer revolutions
   sample every 2 hours (determining: OD₆₂₀, glucose concentration by means of “Accutrend” from Roche, pH offline, dry biomass DBM), storing fermentation supernatant and pellet, (monitoring plasmid stability over the entire process time).
   glucose starting concentration in the fermenter: 13.64 g/l, no regulation of glucose concentration in fermenter but offline determination and corresponding start of Fed-batch—Pro metering system from DASGIP, Jülich with a residual amount of approx. 4 g/l and manual adjustment of metering rate so as not to exceed 5 g/l, if possible.
   process data recording via LabView (National Instruments)
strains: E. coli K-12 LJ110 aroB/pF36 (“PYC”)
   E. coli K-12 LJ110 aroB/pACTCtac (“EV”)

Table 4 below lists the results of the fermentations.
Table 4: Fermentation Results for Obtaining DAH by using Recombinant Pyruvate Carboxylase

Yields (based on glucose used; [mole of product/mole of glucose]) of strains:
LJ110 aroB-/pF36 (Pyc strain) and

LJ110 aroB/-pACYCtac (control with empty vector)

| | | LJ110 aroB-/ | LJ110 aroB-/ |
|---|---|---|---|
| Reactant/products | | pF36 + IPTG | pACTCtac + IPTG |
| Glucose used | [mol] | 0.727 | 0.459 |
| DAH produced | [mol] | 0.074 | 0.018 |
| DAH yield | [mol/mol] | 0.102 | 0.040 |
| Glutamate produced | [mol] | 0.039 | 0.012 |
| Glutamate yield | [mol/mol] | 0.054 | 0.026 |
| Acetate produced | [mol] | 0.065 | 0.365 |
| Acetate yield | [mol/mol] | 0.090 | 0.797 |

The fermentation results reveal that introducing the pyc gene sequence into E. coli resulted in a distinct increase in the yield of DAH. The organisms transformed with the pyc gene sequence had a DAH yield which had increased by at least a factor of 2.5 compared to that of the control organisms which had been transformed with the empty vector (control vector without pyc gene sequence) or whose pyruvate carboxylase had not been induced by addition of IPTG.

1. A process to improve the microbial production of aromatic amino acids and other metabolites of the aromatic amino acid biosynthetic pathway,
which method comprises introducing a pyc gene sequence into a microorganism and allowing said production in said microorganism to occur,
whereby said production is improved.

2. The process of claim 1,
which further comprises amplifying the pyc gene sequence in said microorganism.

3. The process of claim 1,
which further comprises increasing the copy number of the pyc gene sequence in said microorganism.

4. The process of claim 1,
which further comprises increasing gene expression of the pvc gene sequence in said microorganism.

5. The process of claim 1,
wherein said pyc gene sequence is derived from an organism from the group consisting of Corynebacteria, Rhizobia, Brevibacteria, Bacillus, Mycobacteria, Pseudomonas, Rhodopseudomonas, Campylobacter, Methanococcus and Saccharomyces.

6. The process of claim 5,
wherein said pyc gene sequence is derived from Corynebacterium glutamicum.

7. (canceled)

8. The process of claim 1,
wherein said production is of L-phenylalanine, L-tryptophan and/or L-tyrosine.

9. The process of claim 1,
wherein said microorganism is selected from the group consisting of Enterobacteriaceae, Serratia, Bacillus, Corynebacterium and Brevibacterium.

10. The process of claim 1,
wherein said microorganism is Escherichia coli.

11. The process of claim 1,
which further comprises fermenting the microorganism in a medium containing components selected from the group consisting of biotin, IPTG and essential growth substances.

12. (canceled)

13. The process of claim 1,
which further comprises inactivating or switching off at least one PEP-consuming enzyme in said microorganism.

14. The process of claim 13,
which further comprises inactivating or switching off at least one enzyme selected from the group consisting of PEP carboxylases, PEP-dependent sugar phosphotranferases (PTS) and pyruvate kinases in said microorganism.

15. The process of claim 1,
which process further comprises introducing a PEP-independent transport system for glucose uptake into said microorganism.

16. The process of claim 1,
which process further comprises introducing a glucose-facilitator protein (Glf) into said microorganism.

17. The process of claim 1,
which process further comprises introducing a glucose-facilitator protein from Zymomonas mobilis into a microorganism.

18. The process of claim 1
which process further comprises introducing sugar transport genes into said microorganism.

19. The process of claim 1,
which process further comprises introducing a transaldolase and/or transketolase into said microorganism.

20. The process of claim 1,
which process further comprises introducing a transketolase A and/or transketolase B from E. coli into said microorganism.

21. The process of claim 1,
which further comprises deregulating and/or amplifying at least one enzyme selected from the group consisting of DAHP synthase, shikimate kinase, chorismate mutase and prephenate dehydratase.
